(19) United States
(12) Patent Application Publication
(10) Pub. No.: US 2008/0108695 A1
(43) Pub. Date: May 8, 2008

(54) Method of Treating Asthma, Allergic Rhinitis, and Skin Disorders

(75) Inventors: Peter G. Klimko, Fort Worth, TX (US); Clay Beauregard, Burleson, TX (US); Bryon S. Severns, Arlington, TX (US)

Correspondence Address: ALCON, IP LEGAL, TB4-8, 6201 SOUTH FREEWAY, FORT WORTH, TX, 76134, US

(73) Assignee: Alcon Manufacturing Ltd., Fort Worth, TX (US)

(21) Appl. No.: 11/935,457

(22) Filed: Nov. 6, 2007

Related U.S. Application Data
(60) Provisional application No. 60/857,339, filed on Nov. 7, 2006.

(51) Int. Cl. A61K 31/22; A61K 31/351; A61K 31/341; A61P 11/00

(52) U.S. Cl. 514/452; 514/460; 514/546

(57) ABSTRACT

The use of 5,6,7-trihydroxyheptanoic acid and analogs is disclosed for the treatment of asthma, allergic rhinitis, and skin disorders such as allergic dermatitis, contact hypersensitivity, urticaria (hives), rosacea, or psoriasis.

This application claims priority to U.S. Provisional Application, U.S. Ser. No. 60/857,339.

The present invention is directed to the treatment of asthma, allergic rhinitis, and skin disorders. In particular, the present invention is directed toward the use of 5,6,7-trihydroxyheptanoic acid and its analogs to treat these conditions.

### BACKGROUND OF THE INVENTION

Lipoxin A₄ is an anti-inflammatory eicosanoid biosynthesized from arachidonic acid, and is produced locally at inflammation sites via the interaction of neutrophils with platelets or of other leukocytes with epithelial cells. Lipoxin A₄ is believed to act endogenously to resolve inflammation by inhibiting neutrophil influx into inflamed tissue and by inducing macrophage phagocytosis/clearance of activated neutrophils. Lipoxin A₄ binds to at least two receptors with nM affinity. The first is the lipoxin A₄ cognate receptor, called ALXR. This is the same as the formyl peptide receptor FPRL-1. The second receptor is cysLT₁, the high affinity receptor for the cysteinyl leukotriene LTD₄. Lipoxins are thought to function as ALXR agonists and cysLT₁ receptor antagonists [Fronert et al., Am. J. Pathol. 2001, 158(1), 3-8].

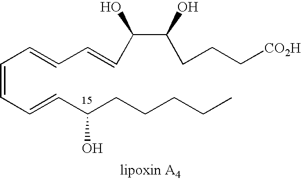

Several researchers have reported that administration of lipoxin A₄ structural analogs inhibit allergen-induced eosinophil infiltration, decrease production of pro-inflammatory allergic mediators like cysteinyl leukotrienes, IL-5, and eotaxin, and reduce tissue edema in several animal models, including: a mouse model of allergic asthma [Levy et al., Nat. Med. 2002, 8(9), 1018-1023]; allergen-induced skin inflammation in mice and guinea pigs [Schottelieus et al., J. Immun. 2002, 169(12), 1029-1036]; and allergen-induced pleurisy in rats [Bandeira-Melo et al., J. Immun. 2000, 164(5), 2267-2271].

Lee et. al. have disclosed that compounds 1 and 2 inhibit LTB₄-induced chemotaxis of neutrophils as potently as lipoxin A₄ [Lee et. al., Biochemical and Biophysical Research Communications 1991, 180(3), 1416-21]. As the authors' stated purpose was to investigate the relationship between this bioassay readout and the structure of lipoxin A₄ analogs that they synthesized, one conclusion could be that compounds 1, 2, and lipoxin A₄ inhibit LTB₄-induced neutrophil chemotaxis by the same mechanism, namely activation of the ALXR.

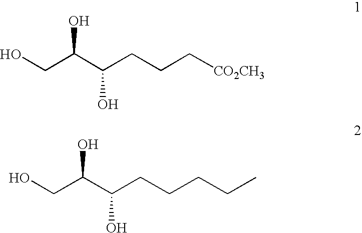

However, this theory may well be invalid. An essential experiment to test this theory would be to ascertain whether the chemotaxis inhibition effect for these three compounds could be blocked by a selective ALXR antibody or small molecule antagonist. This was not performed, since at the time of Lee et al.,'s disclosure neither the ALXR protein nor its associated mRNA had been sequenced [this was accomplished in 1994: J. Exp. Med. 1994, 180(1), 253-260]. An explanation for the neutrophil chemotaxis inhibition displayed by 1, 2, and lipoxin A₄ which is equally consistent with this disclosure would be that 1 and 2 act via leukotriene B₄ receptor antagonism while lipoxin A₄ acts via ALXR agonism and/or perhaps antagonism at the leukotriene D₄ (LTD₄) receptor cysLT₁ [Gronert et al., Am. J. Path. 2000, 158(1), 3-9]. Furthermore it is known that the biological activity of lipoxin A₄ is critically dependent on the presence of a hydroxyl at position 15; oxidation to the carbonyl [Petasis et al., Prostaglandins Leukot. Essent. Fatty Acids 2005, 73(3-4), 301-321] or replacement with a hydrogen [Jozsef et al., Proc. Natl. Acad. Sci. USA 2002, 99(20), 13266-13271] greatly diminishes biological activity. However 1 and 2 lack this hydroxyl, indeed they lack any atoms at all beyond the primary hydroxyl group of their triol array. To the best of our knowledge there have been no subsequent reports on the biological activities of either 1 or 2. Thus absent receptor-linked functional data, one skilled in the art could reasonably doubt that these compounds' inhibition of LTB₄-induced neutrophil chemotaxis is due to ALXR agonism.

### SUMMARY OF THE INVENTION

The present invention is directed to methods for the treatment of asthma, allergic rhinitis, and skin disorders. According to the methods of the present invention, a 5,6,7-trihydroxyheptanoic acid or analog is administered to a patient via oral or inhalation delivery for the treatment of asthma. In a further embodiment of the invention, a 5,6,7-trihydroxyheptanoic acid or analog is administered to a patient via oral or topical nasal delivery for the treatment of allergic rhinitis. In yet another embodiment of this invention, a 5,6,7-trihydroxyheptanoic acid or analog is administered to a patient via topical delivery for the treatment of skin disorders, such as allergic dermatitis, psoriasis, and rosacea.

### DETAILED DESCRIPTION OF THE INVENTION

Unless indicated otherwise, all component amounts are presented on a % (w/v) basis.

According to the methods of the present invention, a composition comprising a compound of formula I is administered to a mammal in need thereof: 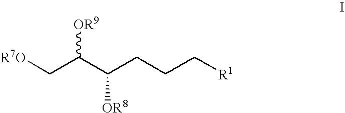
wherein
R¹ is C₂H₅, CO₂R, CONR²R³, CH₂OR⁴, 1,3,4-oxadiazole-2-yl, or CH₂NR⁵R⁶, where:
   R is H, C₁₋₆ straight chain or branched alkyl, C₃₋₆ cycloalkyl, or phenyl, or R¹ is a carboxylate salt of formula CO₂⁻R⁺, where R⁺ is Li⁺, Na⁺, K⁺, or an ammonium moiety of formula ⁺NR¹⁰R¹¹R¹²R¹³;
   R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
   R⁴ is H, C(O)R¹⁴, C₁₋₆ alkyl, C₃₋₆ cycloalkyl, benzyl, or phenyl;
   R⁵, R⁶ are independently H, C(O)R¹⁴, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
   R⁷, R⁸, and R⁹ are independently H, CH₃, C₂H₅, C(O)R¹⁴, or CO₂R¹⁵; or R⁷ and R⁸ or R⁸ and R⁹ together constitute a carbonyl group (C═O), thus forming a cyclic carbonate;
   or OR⁸R¹ together form a cyclic ester (a lactone);
   R¹⁰-R¹³ are independently H or C₁₋₆ alkyl, each alkyl group optionally bearing an OH or OCH₃ substituent;
   R¹⁴ is H, C₁₋₆ alkyl, C₃₋₆ cycloalkyl, benzyl, or phenyl;
   R¹⁵ is C₁₋₆ alkyl, C₃₋₆ cycloalkyl, benzyl, or phenyl; and
   [symbol] indicates that the OR⁹ substituent can be arranged to afford the R or S absolute configuration: 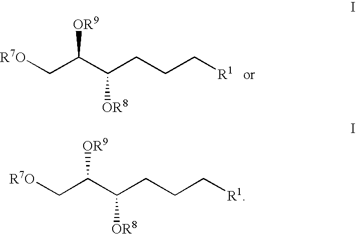
   Preferred compounds of formula I are those wherein:
   R¹ is C₂H₅, CO₂R, CH₂OR⁴, 1,3,4-oxadiazole-2-yl, or a carboxylate salt of formula CO₂⁻R⁺;
   R⁺ is Li⁺, Na⁺, K⁺, or NH₄⁺;
   R is H, CH₃, C₂H₅, n-C₃H₇, or i-C₃H₇;
   R⁴ is H, COCH₃, or CH₃; and
   R⁷, R⁸, R⁹ are independently H, CH₃, or CH₃CO;
   or R⁷ and R⁸ or R⁸ and R⁹ together constitute a carbonyl group (C═O), thus forming a cyclic carbonate;
   or OR⁸R¹ together form a cyclic ester (a lactone).

Among the especially preferred are compounds 1-6. Compound 1 is commercially available from Biomol Research Laboratories, Plymouth Meeting, Pa., and compound 2 can be prepared as detailed in Lee et. al., Biochemical and Biophysical Research Communications 1991, 180(3), 1416-21. Compounds 3-6 can be prepared as described in examples 1-4 below.

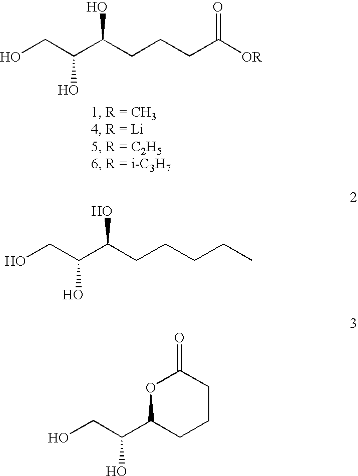

### EXAMPLE 1

### Synthesis of Compound 3

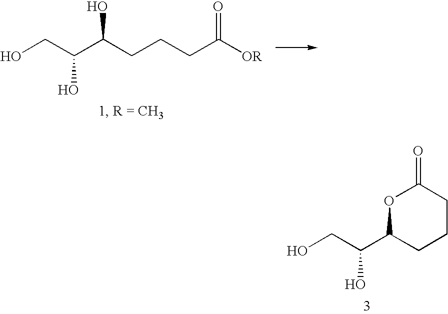

A solution of methyl ester 1 (20 mg, 0.104 mmol) in MeOH (2.1 mL) containing 1 M LiOH (0.5 mL, 0.5 mmol) was heated in a microwave heater at 120° C. for 6 minutes. The reaction was concentrated and the residue was chromatographed on a 10 mm diameter×18 cm tall C18 reverse-phase silica gel column eluting with 7:3 v:v 0.05 M HCl:acetonitrile to afford a crude white solid after concentration (40.9 mg). The solid was rinsed with hot CH₃CN (2×2 mL) and the filtrate was concentrated to afford lactone 3 (7.8 mg, 47%). ¹³C NMR (150 MHz, dmso-d₆) δ 171.12 (C), 79.86 (CH), 72.44 (CH), 62.03 (CH₂), 29.39 (CH₂), 21.67 (CH₂), 17.55 (CH₂).

### EXAMPLE 2

### Synthesis of Compound 4

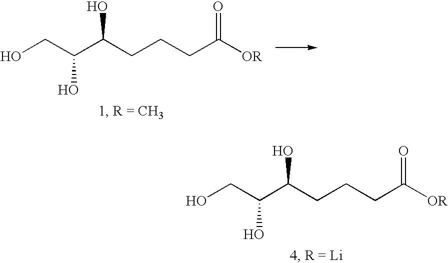

A solution of methyl ester 1 in aqueous MeOH is heated to reflux in the presence of 3 equivalents of lithium hydroxide. After 6 h the reaction is cooled to room temperature and the pH of the solution is adjusted to 6 by the addition of 70-9 mesh sulfonic acid resin MP (commercially available from Novabiochem/EMD Biosciences, 10394 Pacific Center Court, San Diego, Calif. 92121). The solution is filtered through a 0.2 μM poly-terfluoroethylene syringe filter and concentrated to afford the lithium carboxylate 4 as a white solid. ¹H NMR (D₂O, 400 MHz) δ 3.69-3.64 (m, 1H), 3.55-3.47 (m, 3H), 2.16-2.12 (m, 2H), 1.67-1.64 (m, 1H), 1.54-1.48 (m, 2H), 1.38-1.34 (m, 1H). ¹³C NMR (D₂O, 100 MHz) δ 183.46 (C), 74.61 (CH), 71.67 (CH), 62.49 (CH₂), 37.26 (CH₂), 31.55 (CH₂), 22.04 (CH₂).

### EXAMPLE 3

### Synthesis of Compound 8

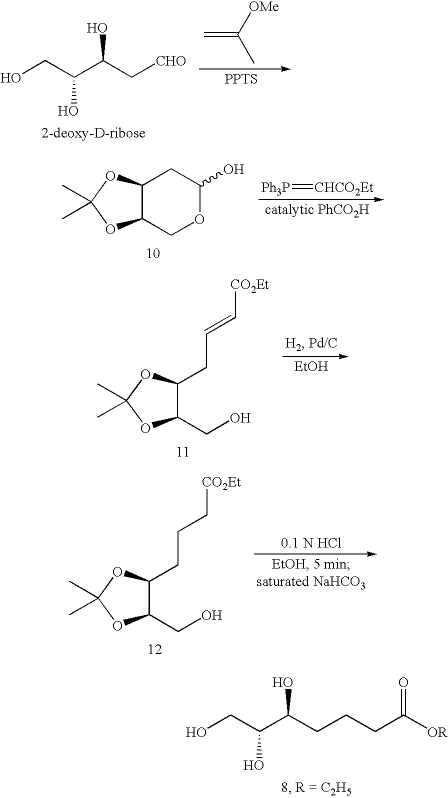

2-deoxy-D-ribose is converted to the acetonide-protected lactol 10 by treatment with 2-methoxypropene and catalytic pyridinium p-toluenesulfonate (PPTS) in ethyl acetate. Wittig reaction with Ph₃P═CHCO₂Et in THF in the presence of catalytic benzoic acid affords enoate 11, which is reduced to 12 under a hydrogen atmosphere in the presence of catalytic Pd/C in ethanol. Deprotection of 12 using 0.1 N HCl in ethanol for 5 minutes, followed by quenching with aqueous NaHCO₃, affords 8 after silica gel chromatographic purification.

### EXAMPLE 4

### Synthesis of Compound 9

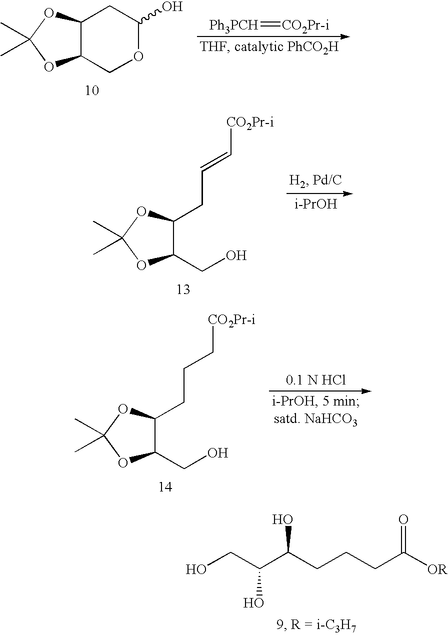

Wittig reaction of lactol 10 with Ph₃P═CHCO₂Et in THF in the presence of catalytic benzoic acid affords enoate 13, which is reduced to 14 under a hydrogen atmosphere in the presence of catalytic Pd/C in isopropanol. Deprotection of 14 using 0.1 N HCl in isopropanol for 5 minutes, followed by quenching with aqueous NaHCO₃, affords 9 after silica gel chromatographic purification.

According to the methods of the present invention, a compound of formula I is administered in a pharmaceutically acceptable carrier. The compositions are formulated in accordance with methods known in the art. Additionally, the compositions may contain a second drug, other than a compound of formula I.

The compositions of the present invention contain a pharmaceutically effective amount of a compound of formula I. As used herein, “a pharmaceutically effective amount” means an amount sufficient to reduce or eliminate asthma, allergic rhinitis, or skin disorder symptoms. Generally, the compositions of the present invention will contain from 0.001 to 5% of a compound of formula I. Preferably, the compositions of the present invention will contain from 0.1 to 5% of a compound of formula I.

The compositions administered according to the present invention may also include various other ingredients, including but not limited to surfactants, tonicity agents, buffers, preservatives, co-solvents and viscosity building agents.

Various tonicity agents may be employed to adjust the tonicity of the composition. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have a tonicity agent in an amount sufficient to cause the final composition to have an acceptable osmolality (generally about 150-450 mOsm, preferably 250-350 mOsm).

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 5.5-8.

Topical products are typically packaged in multidose form. Preservatives are typically required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically will not contain a preservative and will be unpreserved.

The compositions of the present invention can be formulated for various desired dosage forms, depending upon the disorder to be treated. For example, the compositions may be formulated as a composition to be delivered via inhalation using for example a nebulizer, in order to treat asthma. Alternatively, the compositions may be formulated as a topical nasal spray to treat allergic rhinitis. In another embodiment, the compositions may be formulated as a lotion, cream, or ointment to treat skin disorders, such as allergic dermatitis, contact hypersensitivity, urticaria (hives), rosacea, or psoriasis.

Representative formulations are provided below in Examples 6-9.

### EXAMPLE 6

A representative pharmaceutical formulation in nebulized form containing a compound of the invention, useful for the treatment of asthma according to the methods of the present invention, is exemplified below.

| Ingredient | Concentration (% w/v) |
|---|---|
| Compound of formula I | 0.1% |
| Ethanol | 10% |
| Purified Water | 89.9% |

### EXAMPLE 7

A formulation for oral administration containing a compound of the invention, useful for the treatment of asthma according to the methods of the present invention, is exemplified below.

5 mg Capsules

| | mg/capsule |
|---|---|
| Ingredient | (Total Wt. 100 mg) |
| Compound of formula I | 5 |
| Lactose, anhydrous | 55.7 |
| Starch, Sodium carboxy-methyl | 8 |
| Cellulose, microcrystalline | 30 |
| Colloidal silicon dioxide | .5 |
| Magnesium stearate | .8 |

### EXAMPLE 8

A topically administerable nasal solution for the treatment of allergic rhinitis according to the methods of the invention, is exemplified below.

| Ingredient | Concentration (% w/v) |
|---|---|
| Compound of formula I | 0.1% |
| Benzalkonium Chloride | 0.02% |
| Dibasic Sodium Phosphate (Anhydrous) | 0.5% |
| Sodium Chloride | 0.3% |
| Edetate Disodium | 0.01% |
| NaOH/HCl | q.s. to pH 6-8 |
| Purified Water | q.s. to 100% |

### EXAMPLE 9

A topically administerable ointment for the treatment of skin disorders such as allergic dermatitis, contact hypersensitivity, urticaria (hives), rosacea, or psoriasis according to the methods of the invention, is exemplified below.

| Ingredient | Concentration (% w/w) |
|---|---|
| Compound of formula I | 0.1% |
| Cholesterol | 3% |
| Stearyl Alcohol | 3% |
| White Wax | 7.9% |
| White Petrolatum | 86% |

A preferred container for a nasal product is a high-density polyethylene container equipped with a nasal spray pump.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

What is claimed is:

1. A method for the treatment of asthma, allergic rhinitis, or a skin disorder in a mammal, which comprises administering to the mammal a composition comprising a pharmaceutically acceptable carrier and a pharmaceutically effective amount of a compound of formula I: 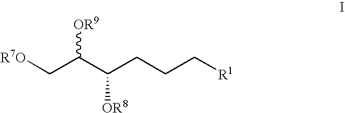
wherein
R¹ is C₂H₅, CO₂R, CONR²R³, CH₂OR⁴, 1,3,4-oxadiazole-2-yl, or CH₂NR⁵R⁶, where:
R is H, C₁₋₆ straight chain or branched alkyl, C₃₋₆ cycloalkyl, or phenyl, or R¹ is a carboxylate salt of formula CO₂⁻R⁺, where R⁺ is Li⁺, Na⁺, K⁺, or an ammonium moiety of formula ⁺NR¹⁰R¹¹R¹²R¹³;
R², R³ are independently H, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁴ is H, C(O)R¹⁴, C₁₋₆ alkyl, C₃₋₆ cycloalkyl, benzyl, or phenyl;
R⁵, R⁶ are independently H, C(O)R¹⁴, C₁₋₆ alkyl, C₃₋₆ cycloalkyl, benzyl, phenyl, OH, OCH₃, or OC₂H₅, provided that at most only one of R², R³ is OH, OCH₃, or OC₂H₅;
R⁷, R⁸, and R⁹ are independently H, CH₃, C₂H₅, C(O)R¹⁴, or CO₂R¹⁵; or R⁷ and R⁸ or R⁸ and R⁹ together constitute a carbonyl group (C═O), thus forming a cyclic carbonate;
or OR⁸R¹ together form a cyclic ester (a lactone);
R¹⁰-R¹³ are independently H or C₁₋₆ alkyl, each alkyl group optionally bearing an OH or OCH₃ substituent;
R¹⁴ is H, C₁₋₆ alkyl, C₃₋₆ cycloalkyl, benzyl, or phenyl;
R¹⁵ is C₁₋₆ alkyl, C₃₋₆ cycloalkyl, benzyl, or phenyl; and [symbol]
indicates that the OR⁹ substituent can be arranged to afford the R or S absolute configuration: 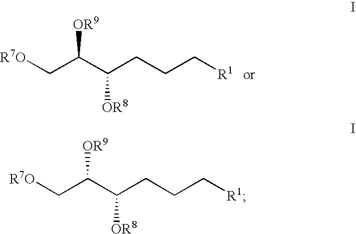
wherein the skin disorder is selected from the group consisting of allergic dermatitis; contact hypersensitivity; urticaria; rosacea; and psoriasis.

2. The method of claim 1 wherein for the compound of formula I:
R¹ is C₂H₅, CO₂R, CH₂OR⁴, 1,3,4-oxadiazole-2-yl, or a carboxylate salt of formula CO₂⁻R⁺;
R⁺ is Li⁺, Na⁺, K⁺, or NH₄⁺;
R is H, CH₃, C₂H₅, n-C₃H₇, or i-C₃H₇;
R⁴ is H, COCH₃, or CH₃; and
R⁷, R⁸, R⁹ are independently H, CH₃, or CH₃CO;
or R⁷ and R⁸ or R⁸ and R⁹ together constitute a carbonyl group (C═O), thus forming a cyclic carbonate;
or OR⁸R¹ together form a cyclic ester (a lactone).

3. The method of claim 2 wherein the compound of formula I has the configuration:

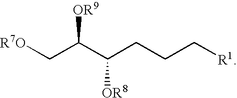

4. The method of claim 2, wherein the compound of formula I has the configuration:

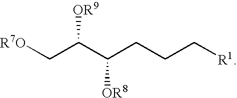

5. The method of claim 3, wherein a compound of formula I is used to treat asthma.

6. The method of claim 3, wherein a compound of formula I is used to allergic rhinitis.

7. The method of claim 3, wherein the compound of formula I is selected from the group consisting of:

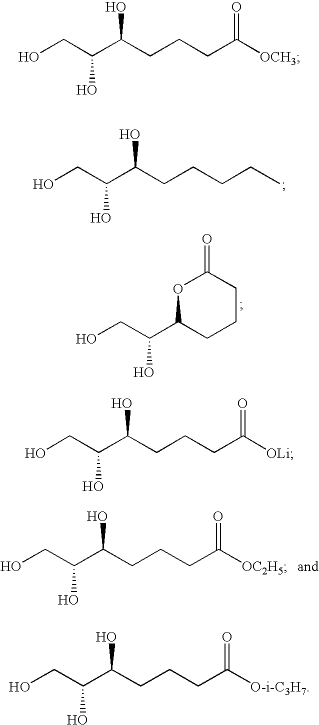

8. The method of claim 7, wherein the pharmaceutically effective amount of compound is from 0.001 to 5% (w/v).

9. The method of claim 8, wherein the pharmaceutically effective amount is from 0.1 to 5% (w/v).

10. The method of claim 1, wherein the pharmaceutically acceptable carrier comprises one or more ingredients selected from the group consisting of surfactants; tonicity agents; buffers; preservatives; co-solvents; and viscosity building agents.

11. The method of claim 1 wherein the method is a method for the treatment of asthma.

12. The method of claim 1 wherein the method is a method for the treatment of allergic rhinitis.

13. The method of claim 1 wherein the method is a method for the treatment of a skin disorder selected from the group consisting of allergic dermatitis; contact hypersensitivity; urticaria; rosacea; and psoriasis.
